(19) United States
(12) Patent Application Publication
(10) Pub. No.: US 2022/0008329 A1
(43) Pub. Date: Jan. 13, 2022

(54) Oral Care Compositions Including Botanicals and Methods for the Same

(71) Applicant: Colgate-Palmolive Company, New York, NY (US)

(72) Inventors: Amy TONG, Guangzhou (CN); Zhaoyu WEN, Guangzhou (CN); Xiao Yi HUANG, Guangzhou (CN)

(73) Assignee: Colgate-Palmolive Company, New York, NY (US)

(21) Appl. No.: 17/413,514

(22) PCT Filed: Dec. 13, 2018
(86) PCT No.: PCT/CN2018/120837

§ 371 Date: Jun. 11, 2021

(51) Int. Cl. A61K 8/9789; A61Q 11/00

(52) U.S. Cl. CPC A61K 8/9789; A61K 2800/48; A61Q 11/00

(57) ABSTRACT

Oral care compositions including one or more botanicals or parts thereof, and methods for preparing the same are provided. The oral care composition may include an orally acceptable vehicle and one or more botanicals or parts thereof. The parts of the botanical may include leaves, fruits, flowers, seeds, petals, or combinations thereof. The oral care composition may also include a thickener capable of or configured to at least partially distribute or suspend the parts of the botanical throughout the orally acceptable vehicle.

### BACKGROUND

Conventional oral care products or compositions often include or incorporate functional, aesthetic, and/or ornamental elements or features to enhance consumer acceptance and/or experience. For example, conventional mouthwashes may often incorporate colors or dyes to provide an aesthetic effect or color that identifies a flavor of the mouthwash that consumers find pleasing, which promotes the use and/or aids in the selection of the mouthwashes.

While the incorporation of the functional, aesthetic, and/or ornamental elements into conventional oral care products have proven to enhance consumer acceptance and distinguish products in the marketplace, the type, selection, or number of these elements that have been successfully incorporated into the oral care products is limited. For example the complexity of making the oral care products and increasingly strict guidelines for safety concerns often limits the elements that are successfully incorporated into the oral care products.

What is needed, then, are improved oral care products incorporating botanicals and/or parts thereof for improved aesthetic and/or therapeutic effects.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

The foregoing and/or other aspects and utilities embodied in the present disclosure may be achieved by providing an oral care composition including an orally acceptable vehicle and one or more botanicals and/or parts thereof. In at least one implementation, the oral care composition includes parts of a botanical.

In at least one implementation, a genus of the botanical may be selected from Origanum, Thymus, Lavandula, Salvia, Melissa, Cuminum, Pelroselinum, Calendula, Tageles, Boswellia, Sambucus, Copaifera, Curcuma, Allium, Symphylum, Punica, Eulerpe, Sophora, Rheum, Fagopyrum, Camellia, Coplis, Hydraslis, Mahonia, Phellodendron, Berberis, Xanthorhiza, Lonicera, Vaccinium, Cinnamomum, VlZlS, Terminalia, Pinus, Albizia, Melia, Salvadora, Paullinia, Piper, Syzygium, Commiphora, Juglans, Sculellaria, Magnolia, or combinations thereof.

In at least one implementation, the botanical may include a botanical source selected from roses, gardenias, jasmine, calendula, honeysuckle, cedar wood, aspen bark, willow bark, brahmi, green tea, grapes, pomegranate, echinacea, Centella asiatica, elderflower, Irish moss, mallow, soap bark, yucca, clary sage, oregano, thyme, or combinations thereof.

In at least one implementation, the botanical source may include gardenias.

In at least one implementation, the botanical source may include jasmine.

In at least one implementation, the oral care composition may be a mouthwash.

In at least one implementation, the orally acceptable vehicle may include a thickener configured to at least partially distribute and/or suspend the parts of the botanical throughout the orally acceptable vehicle.

In at least one implementation, the thickener may include xanthum gum.

In at least one implementation, the orally acceptable vehicle may be free or substantially free of any thickeners.

In at least one implementation, the parts of the botanical may include one or more of the following: leaves, fruits, flowers, seeds, petals, or combination thereof.

In at least one implementation, the parts of the botanical may include petals.

The foregoing and/or other aspects and utilities embodied in the present disclosure may be achieved by providing a method for preparing any one of the oral care compositions disclosed herein. The method may include contacting the parts of the botanical with the orally acceptable vehicle.

In at least one implementation, the method may further include at least partially distributing the parts of the botanical throughout the orally acceptable vehicle. At least partially distributing the parts of the botanical may include adding a thickener to the orally acceptable vehicle. In at least one implementation, the thickener may include xanthum gum.

The foregoing and/or other aspects and utilities embodied in the present disclosure may be achieved by providing a method for treating or reducing one or more oral conditions in a human in need thereof. The method may include contacting any one of the oral care compositions disclosed herein with surfaces of an oral cavity of the human. In at least one implementation, the method may include contacting the oral care composition with the surfaces of the oral cavity at least once per day, optionally, at least twice per day.

Further areas of applicability of the present disclosure will become apparent from the detailed description provided hereinafter. It should be understood that the detailed description and specific examples, while indicating some typical aspects of the disclosure, are intended for purposes of illustration only and are not intended to limit the scope of the disclosure.

### DETAILED DESCRIPTION

The following description of various aspect(s) is merely exemplary in nature and is in no way intended to limit the disclosure, its application, or uses.

As used throughout this disclosure, ranges are used as shorthand for describing each and every value that is within the range. It should be appreciated and understood that the description in a range format is merely for convenience and brevity, and should not be construed as an inflexible limitation on the scope of any embodiments or implementations disclosed herein. Accordingly, the disclosed range should be construed to have specifically disclosed all the possible subranges as well as individual numerical values within that range. As such, any value within the range may be selected as the terminus of the range. For example, description of a range such as from 1 to 5 should be considered to have specifically disclosed subranges such as from 1.5 to 3, from 1 to 4.5, from 2 to 5, from 3.1 to 5, etc., as well as individual numbers within that range, for example, 1, 2, 3, 3.2, 4, 5, etc. This applies regardless of the breadth of the range.

Unless otherwise specified, all percentages and amounts expressed herein and elsewhere in the specification should be understood to refer to percentages by weight. The amounts given are based on the active weight of the material.

Additionally, all numerical values are “about” or “approximately” the indicated value, and take into account experimental error and variations that would be expected by a person having ordinary skill in the art. It should be appreciated that all numerical values and ranges disclosed herein are approximate values and ranges, whether “about” is used in conjunction therewith. It should also be appreciated that the term “about,” as used herein, in conjunction with a numeral refers to a value that may be ±0.01% (inclusive), ±0.1% (inclusive), ±0.5% (inclusive), ±1% (inclusive) of that numeral, ±2% (inclusive) of that numeral, ±3% (inclusive) of that numeral, ±5% (inclusive) of that numeral, ±10% (inclusive) of that numeral, or ±15% (inclusive) of that numeral. It should further be appreciated that when a numerical range is disclosed herein, any numerical value falling within the range is also specifically disclosed.

As used herein, “free” or “substantially free” of a material may refer to a composition, component, or phase where the material is present in an amount of less than 10.0 weight %, less than 5.0 weight %, less than 3.0 weight %, less than 1.0 weight %, less than 0.1 weight %, less than 0.05 weight %, less than 0.01 weight %, less than 0.005 weight %, or less than 0.0001 weight % based on a total weight of the composition, component, or phase.

All references cited herein are hereby incorporated by reference in their entireties. In the event of a conflict in a definition in the present disclosure and that of a cited reference, the present disclosure controls.

### Compositions

Compositions disclosed herein may be or include an oral care product or an oral care composition thereof. For example, the compositions disclosed herein may be an oral care product, such as a mouthwash, including the oral care composition, or the oral care composition thereof. In at least one implementation, the oral care composition may include an orally acceptable vehicle or carrier and one or more botanicals and/or parts thereof. For example, the oral care composition may include an orally acceptable vehicle and one or more botanicals. In another example, the oral care composition may include an orally acceptable vehicle and one or more parts of a botanical or one or more parts of a plurality of botanicals. In yet another example, the oral care composition may include an orally acceptable vehicle, one or more botanicals, and one or more parts of a botanical or one or more parts of a plurality of botanicals. In at least one implementation, the oral care compositions disclosed herein are not intended to be part of a diet and are not intended to be ingested. For example, any therapeutic benefits disclosed herein may be provided by contact with surfaces of the oral cavity and without ingestion.

The one or more botanicals and/or parts thereof may be mixed, combined, disposed, suspended, or otherwise contacted with the orally acceptable vehicle. In at least one implementation, the one or more botanicals and/or parts thereof may generally be evenly distributed throughout the orally acceptable vehicle. For example, the one or more botanicals and/or parts thereof may be suspended and substantially evenly distributed throughout the orally acceptable vehicle. In another implementation, the one or more botanicals and/or parts thereof may be disposed in the orally acceptable vehicle and the one or more botanicals and/or parts thereof may be generally concentrated in a portion of the orally acceptable vehicle. For example, the orally acceptable vehicle may be disposed in a container or vessel, and the one or more botanicals and/or parts thereof may be disposed in the orally acceptable vehicle and settled at a bottom portion of the container (e.g., settling via density). In yet another implementation, at least a portion of the one or more botanicals and/or parts thereof may be generally distributed throughout the orally acceptable vehicle and at least another portion may be generally concentrated in the orally acceptable vehicle.

Botanicals and/or Parts Thereof

The one or more botanicals and/or parts thereof disposed in the orally acceptable vehicle may be a therapeutic agent, a cosmetic agent, and/or a decorative/aesthetic agent. In at least one implementation, the botanicals and/or parts thereof disposed in the orally acceptable vehicle may be a therapeutic agent capable of or configured to provide one or more therapeutic benefits. Illustrative therapeutic benefits may be or include, but are not limited to, anti-bleeding, anti-inflammatory, anti-bacteria, wound-healing, anti-oxidation, deodorization, sensorial related to oral comfort (e.g., oral hydration, gum soothing, etc.), or the like, or any combination thereof.

In another implementation, the botanicals and/or parts thereof disposed in the orally acceptable vehicle may be a cosmetic agent and/or a decorative agent, and may not or does not provide any therapeutic benefits. For example, the botanicals and/or parts thereof may not provide any therapeutic benefits and may instead communicate or inform a consumer of one or more properties of the oral care product or the oral care composition thereof. The one or more properties of the oral care product or the oral care composition thereof that may be communicated to the consumer may be or include, but are not limited to, flavor, scent, brand, quality, emotional perception, mouthfeel, or the like, or any combination thereof. In another example, botanicals and/or parts thereof may not provide any therapeutic benefits and may instead communicate or inform a consumer of the presence of one or more components in the oral care product or the oral care composition thereof. For example, the one or more botanicals and/or parts thereof may communicate to the consumer of the presence of one or more elements or components from nature, one or more natural elements, and/or natural ingredients in the oral care product or the oral care composition thereof. In another example, the one or more botanicals and/or parts thereof may be a decorative agent capable of or configured to provide an aesthetically pleasing and/or consumer acceptable decorative oral care composition that enhances consumer experience and/or acceptance.

In yet another implementation, the one or more botanicals and/or parts thereof may provide at least one therapeutic benefit and may further inform the consumer of one or more properties of and/or the presence of one or more components in the oral care composition. For example, the one or more botanicals and/or parts thereof may provide a therapeutic benefit and communicate to the consumer of one or more properties of the oral care product or the oral care composition thereof.

The one or more botanicals and/or parts thereof may be provided by any suitable botanical sources. Illustrative botanical sources may be or include, but are not limited to, any one or more of the botanicals in the Kingdom Plantae, which may include any one or more botanical sources from any one or more of the following genus: Origanum, Thymus, Lavandula, Salvia, Melissa, Cuminum, Pelroselinum, Calendula, Tageles, Bowellia, Sambucus, Copaifera, Curcuma, Allium, Symphylum, Punica, Eulerpe, Sophora, Rheum, Fagopyrum, Camellia, Coplis, Hydraslis, Mahonia, Phellodendron, Berberis, Xanthorhiza, Lonicera, Vaccinium, Cinnamomum, VlZlS, Terminalia, Pinus, Albizia, Melia, Salvadora, Paullinia, Piper, Syzygium, Commiphora, Juglans, Sculellaria, and Magnolia. Illustrative botanical sources may also be or include, but are not limited to, roses, gardenias, jasmine, calendula, honeysuckle, cedar wood, aspen bark, willow bark, brahmi (Bacopa monnieri), green tea, grapes, pomegranate, echinacea, Centella asiatica, elderflower, Irish moss, mallow, soap bark, yucca, clary sage, oregano, thyme, or the like, or any combinations thereof.

The parts of the botanicals that may be utilized in the oral care product or the oral care composition thereof may include, but is not limited to, any one or more of a root, a stem, a leaf, a fruit, a flower, a seed of the botanical, a petal, a part or portion of any one of the foregoing, or the like, or any combination thereof. In at least on exemplary implementation, the parts of the botanicals utilized in the oral care product or the oral care composition thereof includes petals from the flower of the botanical.

The amount of the one or more botanicals and/or parts thereof present in the oral care product or the oral care composition thereof may be from about 0.001 weight % to about 30 weight %, based on a dried, rehydrated, or fresh weight of the one or more botanicals and/or parts thereof relative to a total weight of the oral care product or the oral care composition thereof. For example, the amount of the one or more botanicals and/or parts thereof present in the oral care product or the oral care composition thereof may be from about 0.001 weight %, 0.005 weight %, about 0.01 weight %, about 0.05 weight %, about 0.1 weight %, about 0.5 weight %, about 1 weight %, about 5 weight %, about 10 weight %, or about 15 weight % to about 20 weight %, about 25 weight %, or about 30 weight %, based on a dried, rehydrated, or fresh weight of the one or more botanicals and/or parts thereof relative to a total weight of the oral care product or the oral care composition thereof.

### Orally Acceptable Vehicle

As discussed above, the compositions disclosed herein may be an oral care composition. The oral care composition may form at least a portion of or be used in one or more oral care products. The oral care composition may be, may include, or be combined with an orally acceptable vehicle to form the oral care product (e.g., mouthwash). Illustrative oral care products may include, but are not limited to, a toothpaste (dentifrice), a prophylactic paste, a tooth powder, a tooth polish, a tooth gel (e.g., a whitening gel), a chewing gum, a lozenge, a mouthwash, a whitening strip, a paint-on gel, varnish, veneer, and tube, syringe or dental tray comprising a gel or paste, or a gel or paste coated on an application support such as dental floss or a toothbrush (e.g., a manual, electric, sound, a combination thereof or ultrasound toothbrush), a tablet (e.g., effervescing tablets), granules, a caplet, wafers, films, beads, or the like. In a preferred implementation, the oral care composition may be or may form at least a portion of a mouthwash.

In at least one implementation, the one or more botanicals and/or parts thereof may be dispersed and/or disposed in the orally acceptable vehicle. As used herein, the expression “orally acceptable vehicle” may refer to a suitable vehicle, ingredient, or combination of ingredients, which can be used to suspend, hold, mobilize, or otherwise contain the one or more botanicals and/or parts thereof in a safe and effective manner. For example, the orally acceptable vehicle may be a suitable solvent, and the one or more botanicals and/or parts thereof may be dispersed, dissolved, mixed, or otherwise contacted with the suitable solvent to prepare or form the oral care product or the oral care composition thereof. Illustrative solvents may be or include, but are not limited to, water, such as deionized or demineralized water, ethanol, methanol, isopropanol, butanol, water, acetone, methyl ethyl ketone, ethyl acetate, butyl acetate, cyclohexanone, cyclohexane, methylene chloride, chloroform, carbon tetrachloride, trichloroethylene, perchloroethylene, methyl glycol acetate, toluene, benzene, diethyl ether, benzyl alcohol, or the like, and mixtures or combinations thereof. In a preferred implementation, the orally acceptable vehicle includes water, more preferably demineralized water.

It should be appreciated that the orally acceptable vehicle may include materials such as, but not limited to, one or more antibacterial agents, fluoride ion sources, anticalculus agents, buffers, flavoring agents, sources of peroxide (e.g., hydrogen peroxide), alkali metal bicarbonate salts, thickening materials, antimicrobial agents, humectants, additional water, surfactants, titanium dioxide, cooling agents, coloring agents, or the like, or mixtures or combinations thereof. In at least one implementation, the one or more botanicals and/or parts thereof or the orally acceptable vehicle thereof may exclude any one or more of the aforementioned materials.

The orally acceptable vehicle may make up the balance of the oral care product or the oral care composition thereof. In at least one implementation, the orally acceptable vehicle (e.g., water) may be present in an amount of at least 60 weight %, at least 62 weight %, at least 64 weight %, at least 66 weight %, at least 68 weight %, at least 70 weight %, at least 72 weight %, at least 74 weight %, at least 76 weight %, at least 78 weight %, at least 80 weight %, at least 82 weight %, at least 84 weight %, at least 86 weight %, at least 88 weight %, at least 90 weight %, at least 92 weight %, at least 94 weight %, at least 96 weight %, at least 98 weight %, at least 99 weight %, or more, based on a total weight of the oral care product or the oral care composition thereof.

### Flavoring Agents

The oral care composition or the orally acceptable vehicle thereof may also include one or more flavoring agents. Illustrative flavoring agents that may be utilized in the film forming composition may be or include, but are not limited to, essential oils and various flavoring aldehydes, esters, alcohols, and similar materials, as well as sweeteners such as sodium saccharin and/or sorbitol, peppermint flavor (e.g., OPTAMINT®), FREEZESTORM™, which is commercially available from Firmenich of Geneva, Switzerland, or the like, or mixtures or combinations thereof. Illustrative essential oils may include, but are not limited to, oils of spearmint, peppermint, wintergreen, sassafras, clove, sage, eucalyptus, marjoram, cinnamon, lemon, lime, grapefruit, and orange. Also useful are chemicals such as menthol, carvone, anethole, or the like, or mixtures or combinations thereof.

The amount or concentration of the one or more flavoring agents present in the oral care product or the film forming composition thereof may vary widely. In at least one implementation, the amount of the one or more flavoring agents present may be from about 0.01 weight % to about 50 weight %, based on a total weight of the oral care product or the film forming composition thereof. For example, the amount of the one or more flavoring agents present may be from about 0.01 weight %, about 1 weight %, about 5 weight %, about 10 weight %, about 15 weight %, about 20 weight %, or about 25 weight % to about 30 weight %, about 35 weight %, about 40 weight %, about 45 weight %, or about 50 weight %.

### Fluoride Ion Source

In at least one implementation, the oral care composition or the orally acceptable vehicle thereof may be free or substantially free of fluoride (e.g., soluble fluoride salts). In another implementation, the oral care products and/or the oral care composition thereof may further include fluoride, such as fluoride from one or more fluoride ion sources (e.g., soluble fluoride salts). A wide variety of fluoride ion-yielding materials may be employed as sources of soluble fluoride. Examples of suitable fluoride ion-yielding materials may be found in U.S. Pat. No. 3,535,421 to Briner et al., U.S. Pat. No. 4,885,155 to Parran, Jr. et al., and U.S. Pat. No. 3,678,154 to Widder et al., the disclosures of which are incorporated herein by reference. Illustrative fluoride ion sources include, but are not limited to, fluoride, stannous fluoride, sodium fluoride, potassium fluoride, sodium monofluorophosphate, fluorosilicate salts, such as sodium fluorosilicate and ammonium fluorosilicate, amine fluoride, ammonium fluoride, and combinations thereof. In a typical implementation, the fluoride ion source includes sodium monofluorophosphate. The amount of the fluoride ion source in the oral care composition may be greater than 0 weight % and less than 0.8 wt %, less than 0.7 wt %, less than 0.6 wt %, less than 0.5 wt %, less than 0.4 wt %, less than 0.3 wt %, less than 0.2 wt %, less than 0.1 wt %, less than 0.08 wt %, less than 0.06 wt %, less than 0.05 wt %, or less than 0.04 wt %. The fluoride ion sources may be present in an amount sufficient to provide a total of about 100 to about 20,000 ppm, about 200 to about 5,000 ppm, or about 500 to about 2,500 ppm fluoride ions.

Antimicrobial Agents and/or Preservatives

The oral care composition or the orally acceptable vehicle thereof may also include one or more antimicrobial agents and/or one or more preservatives. Illustrative antimicrobial agents may include, but are not limited to, methylisothiazolinone (MIT), sodium benzoate, potassium sorbate, halogenated diphenyl ether (e.g. triclosan), herbal extracts and essential oils (e.g., rosemary extract, tea extract, magnolia extract, thymol, menthol, eucalyptol, geraniol, carvacrol, citral, hinokitol, catechol, methyl salicylate, epigallocatechin gallate, epigallocatechin, gallic acid, miswak extract, sea-buckthorn extract), bisguanide antiseptics (e.g., chlorhexidine, alexidine or octenidine), quaternary ammonium compounds (e.g., cetylpyridinium chloride (CPC), benzalkonium chloride, tetradecylpyridinium chloride (TPC), N-tetradecyl-4-ethylpyridinium chloride (TDEPC), phenolic antiseptics, hexetidine, octenidine, sanguinarine, povidone iodine, delmopinol, salifluor, other metal ions (e.g., stannous salts, copper salts, iron salts), sanguinarine, propolis and oxygenating agents (e.g., hydrogen peroxide, buffered sodium peroxyborate or peroxycarbonate), phthalic acid and its salts, monoperthalic acid and its salts and esters, ascorbyl stearate, oleoyl sarcosine, alkyl sulfate, dioctyl sulfosuccinate, salicylanilide, domiphen bromide, delmopinol, octapinol, and other piperidino derivatives, nicin preparations, chlorite salts; and mixtures of any of the foregoing. In an exemplary implementation, the antibacterial agent includes cetylpyridinium chloride (CPC).

### Humectants

The oral care composition or the orally acceptable vehicle thereof may include one or more humectants. The humectants may be capable of or configured to reduce evaporation and lower water activity. It should be appreciated that the humectants may also be capable of imparting desirable sweetness or flavor to the oral care composition or the orally acceptable vehicle thereof. Illustrative humectants may include, but are not limited to polyhydric alcohols, such as glycerin, sorbitol, xylitol, propylene glycol, as well as other polyols, and mixtures thereof.

### Surfactants

The oral care composition or the orally acceptable vehicle thereof may include one or more surfactants. For example, the oral care composition or the orally acceptable vehicle thereof may include one or more anionic surfactants, one or more cationic surfactants, one or more zwitterionic surfactants, one or more nonionic surfactants, or mixtures thereof. Examples of suitable surfactants may be found in U.S. Pat. No. 3,959,458 to Agricola et al., U.S. Pat. No. 3,937,807 to Haack, and U.S. Pat. No. 4,051,234 to Gieske el al., the disclosures of which are incorporated herein by reference in their entirety to the extent they are consistent with the present disclosure.

In at least one implementation, the oral care composition or the orally acceptable vehicle thereof may include at least one anionic surfactant. Illustrative anionic surfactants may include, but are not limited to, water-soluble salts of higher fatty acid monoglyceride monosulfates, such as a sodium salt of a monosulfated monoglyceride of hydrogenated coconut oil fatty acids, such as sodium N-methyl-N-cocoyl taurate, sodium cocomonoglyceride sulfate. Illustrative anionic surfactants may also include higher alkyl sulfates. As used herein, “higher alkyl” refers to C₆₋₃₀ alkyl. For example, in a preferred implementation the anionic surfactant is sodium lauryl sulfate. The anionic surfactants may also include higher alkyl-ether sulfates. For example, the anionic surfactants may have a formula CH₃(CH₂)ₘCH₂(OCH₂CH)ₙOSO₃X, where m is 6-16, n is 1-6, and X is Na or K. In an exemplary implementation, m is 10, and n is 2, 3, or 4, and X is Na or K. For example, the anionic surfactant may be sodium laureth-2 sulfate (CH₃(CH₂)₁₀CH₂(OCH₂CH₂)₂OSO₃Na). In another implementation, the anionic surfactant may include higher alkyl aryl sulfonates, such as sodium dodecyl benzene sulfonate (sodium lauryl benzene sulfonate), and higher alkyl sulfoacetates, such as sodium lauryl sulfoacetate (dodecyl sodium sulfoacetate), higher fatty acid esters of 1,2 dihydroxy propane sulfonate, sulfolaurate (N-2-ethyl laurate potassium sulfoacetamide) and sodium lauryl sarcosinate. In an exemplary implementation, the anionic surfactant is a water soluble salt of alkyl sulfates having from 10 to 18 carbon atoms in the alkyl radical and water soluble salts of sulfonated monoglycerides of fatty acids having from 10 to 18 carbon atoms. For example, the anionic surfactant may be or include, sodium lauryl sulfate, sodium lauroyl sarcosinate, sodium coconut monoglyceride sulfonates, or the like, and mixtures thereof.

In at least one implementation, the oral care composition or the orally acceptable vehicle thereof may include at least one nonionic surfactant. The nonionic surfactant may function as an emulsifier. Illustrative nonionic surfactants may include, but are not limited to, Poloxamers or the like. For example, the nonionic surfactants may include polysorbate 20, poloxamer 407, Poloxamer 338, or the like, or mixtures thereof. The nonionic surfactants may also include, but are not limited to, ethoxylated and hydrogenated ethoxylated castor oils, such as those commonly designated as PEG NN castor oil or PEG NN hydrogenated castor oil, where “NN” designates the number of ethylene oxide units polymerized onto the castor oil to form the nonionic surfactant. For example, the nonionic surfactants may be or include PEG 16, 20, 25, 30, 40, 50, 60, 80, 100, 200, and combinations thereof. In a preferred implementation, the nonionic surfactant is Poloxamer 407.

The amount of any one or more of the surfactants in the oral care composition or the orally acceptable vehicle thereof may be from about 0.10 wt %, about 0.20 wt %, about 0.30 wt %, about 0.40 wt %, about 0.45 wt %, about 0.49 wt %, or about 0.50 wt % to about 0.51 wt %, about 0.55 wt %, about 0.60 wt %, about 0.65 wt %, about 0.70 wt %, about 0.75 wt %, about 0.80 wt %, or greater. In another example, the amount of any one or more of the surfactants in the oral care composition or the orally acceptable vehicle thereof may be about 0.10 wt % to about 0.90 wt %, about 0.20 wt % to about 0.80 wt %, about 0.30 wt % to about 0.70 wt %, about 0.40 wt % to about 0.60 wt %, about 0.45 wt % to about 0.55 wt %, or about 0.50 wt % to about 0.51 wt %. In yet another example, the amount of any one or more of the surfactants in the oral care composition or the orally acceptable vehicle thereof may be greater than 0.10 wt %, greater than 0.20 wt %, greater than 0.30 wt %, greater than 0.40 wt %, greater than 0.45 wt %, greater than 0.49 wt %, or greater than 0.50 wt %.

### Thickeners

In at least one implementation, the oral care composition or the orally acceptable vehicle thereof may be free or substantially free of any thickeners or thickening agents. As used herein, “free” or “substantially free” may refer to a composition, component, or phase that contains less than 10.0 wt %, less than 5.0 wt %, less than 3.0 wt %, less than 1.0 wt %, less than 0.1 wt %, less than 0.05 wt %, less than 0.01 wt %, less than 0.005 wt %, or less than 0.0001 wt %, based on a total weight of the oral care composition or the orally acceptable vehicle thereof.

In another implementation, the oral care composition or the orally acceptable vehicle thereof may include one or more thickeners or thickening agents capable of or configured to at least partially disperse, suspend, or evenly distribute the one or more botanicals and/or parts thereof throughout the orally acceptable vehicle. In at least one implementation, the thickener may be or include one or more polymers. Illustrative polymers may be or include, but are not limited to, polyvinylmethyl ether maleic acid copolymers and/or polysaccharides, such as cellulose derivatives, polysaccharide gums, or the like, or combinations thereof. The cellulose derivatives may include, but are not limited to, carboxymethyl cellulose. The polysaccharide gums may include, but are not limited to, xanthum gum, carrageenan gum, or the like, or mixtures thereof.

### Additional Ingredients

It should be appreciated by one having ordinary skill in the art, that the oral care products and/or the oral care compositions thereof may include other additional ingredients/components. For example, the oral care products and/or the oral care compositions thereof may include desensitizing agents, viscosity modifiers, diluents, surface active agents (e.g., emulsifiers, foam modulators, etc.), pH modifying agents (e.g., acids and bases), mouth feel agents, sweetening agents, colorants/dies, preservatives, or the like, or combinations or mixtures thereof. It should further be appreciated by one having ordinary skill in the art that while general attributes of each of the above categories of materials may differ, there may be some common attributes and any given material may serve multiple purposes within two or more of such categories of materials.

All ingredients for use in the compositions described herein should be orally acceptable. As used herein, “orally acceptable” may refer to any ingredient that is present in a composition as described in an amount and form that does not render the composition unsafe for use in the oral cavity.

### Method

The present disclosure may provide a method for preparing an oral care product or an oral care composition thereof. The method may include preparing an orally acceptable vehicle for the oral care composition. The method may also include contacting one or more botanicals and/or parts thereof with the orally acceptable vehicle. In at least one implementation, the one or more botanicals and/or parts thereof may be heated or placed in a heated medium, such as water, to solubilize or facilitate the solubilization of a portion of any anthocyanin contained in the botanicals and/or the parts thereof. In at least one implementation, the method may include suspending or substantially distributing the one or more botanicals and/or parts thereof in the orally acceptable vehicle. Suspending or substantially distributing the one or more botanicals and/or parts thereof in the orally acceptable vehicle may include adding one or more thickeners or thickening agents to the orally acceptable vehicle. In another implementation, the oral care composition may be free or substantially free of any thickeners or thickening agents; and thus, the method may exclude adding any thickeners or thickening agents.

The present disclosure may provide a method for treating, reducing, or ameliorating one or more oral conditions in a user, human, or animal subject in need thereof with the oral care compositions including the one or more botanicals and/or parts thereof. As used herein “animal subject” may include higher order non-human mammals such as canines, felines, and horses. The one or more oral conditions may include any oral conditions treatable with any one or more therapeutic benefits of the botanicals and/or parts thereof. As discussed above, the one or more therapeutic benefits may be or include, but are not limited to, anti-bleeding, anti-inflammatory, anti-bacteria, wound-healing, anti-oxidation, deodorization, sensorial related to oral comfort (e.g., oral hydration, gum soothing, etc.), or the like, or any combination thereof. The method may include contacting the oral care composition with surfaces of the oral cavity. For example, the method may include rinsing, swishing, or gargling the oral care composition in the oral cavity to contact the oral care composition with surfaces of the oral cavity. The method may include contacting the oral care composition with surfaces of the oral cavity at least once per day, at least twice per day, at least three times per day, or more.

The present disclosure may provide a method for communicating or informing a consumer of one or more properties of an oral care product or an oral care composition thereof. The method may include adding one or more botanicals and/or parts thereof to an orally acceptable vehicle of the oral care product or the oral care composition thereof. The method may also include disposing the oral care product or the oral care composition thereof in a container. In at least one implementation, the container may be at least partially transparent such that the one or more botanicals and/or parts thereof are visible. In another implementation, the container may be opaque such that the one or more botanicals and/or parts thereof are not visible.

The present disclosure may provide a method for communicating or informing a consumer of the presence of one or more components in an oral care product or an oral care composition thereof. The method may include adding one or more botanicals and/or parts thereof to an orally acceptable vehicle of the oral care product or the oral care composition thereof. The method may also include disposing the oral care product or the oral care composition thereof in a container. In at least one implementation, the container may be at least partially transparent such that the one or more botanicals and/or parts thereof are visible. In another implementation, the container may be opaque such that the one or more botanicals and/or parts thereof are not visible.

### EXAMPLES

The examples and other implementations described herein are exemplary and not intended to be limiting in describing the full scope of compositions and methods of this disclosure. Equivalent changes, modifications and variations of specific implementations, materials, compositions and methods may be made within the scope of the present disclosure, with substantially similar results.

### Example 1

Parts of a botanical were prepared for addition to an oral care composition. Particularly, dried petals of a calendula were prepared for addition to a mouthwash. To prepare parts of the botanical, the petals of the calendula were placed in water maintained at about 70° C. to about 80° C. to solubilize or facilitate the solubilization of at least a portion the anthocyanin contained in the petals of the calendula. Facilitating the solubilization of the anthocyanin contained in the petals of the calendula in the heated water reduces the amount of the anthocyanin contained in the petals; and thus, reduces any fading of the color during the shelf life of the oral care composition. Placing the dried petals of the calendula in the heated water (about 70° C. to about 80° C.) also increases the mobility and flexibility of the petals, thereby allowing them to be more easily manipulated (e.g., unfolding, separating, etc.). It should be appreciated that prior to heating the petals in the water, the petals of the calendula had a rich deep orange color and were not translucent. After heating the petals of the calendula in the water, the petals of the calendula had a light yellow color and were relatively more translucent as compared to the petals prior to heating. After heating the petals in the water, the petals were dried on a filter paper. After hydration in the heated water, the petals of the calendula were also smoother, flattened, and not bundled up.

### Example 2

A shaking oral care composition (1) and a suspending oral care composition (2) were prepared by combining the ingredients/components according to Table 1. Particularly, each of the shaking and suspending oral care compositions (1)(2) were prepared by contacting and mixing each of the components of Table 1 with one another. The petals utilized were the petals of the calendula prepared in Example 1.

TABLE 1
Oral Care Compositions (1) and (2)

| Ingredient | 1 | 2 |
|---|---|---|
| Demineralized Water (wt %) | 79.073 | 77.096 |
| Propylene Glycol (wt %) | 6.996 | 6.821 |
| Flavoring Agents (wt %) | 0.3038 | 0.2962 |
| Humectant (wt %) | 5.4967 | 5.3593 |
| Sodium Fluoride (wt %) | 0.0500 | 0.0487 |
| Cetylpyridinium Chloride (wt %) | 0.0750 | 0.0731 |
| Preservative (wt %) | 0.0500 | 0.0487 |
| Surfactant (wt %) | 0.400 | 0.390 |
| Colorants/Dies (wt %) | 0.000230 | 0.000224 |
| Glycerin (wt %) | 7.496 | 9.308 |
| Petals (wt %) | 0.0600 | 0.0585 |
| Xanthum Gum (wt %) | 0.0 | 0.500 |
| Total | 100.00 | 100.00 |

The petals in the shaking oral care composition (1) settled to the bottom, but were readily observable as the oral care composition (1) was sufficiently transparent to allow the visibility of the petals. The suspending oral care composition (2) exhibited an even distribution of the petals throughout. Particularly, the inclusion of a thickener, namely xanthum gum, into the oral care composition (2) provided a generally uniform distribution of the petals throughout the suspending oral care composition (2).

### Example 3

A shaking oral care composition (1) and a suspending oral care composition (2) were similarly prepared by combining the ingredients/components according to Table 1. The petals utilized were dried petals of a jasmine. The petals of the jasmine were not subjected to any preparation procedures prior to combining with the other components of the oral care compositions (1) (2), as jasmine petals have a generally white color, which indicates a relatively low amount of anthocyanin. Similar to the petals of the calendula, the petals of the jasmine in the shaking oral care composition (1) settled to the bottom, but were readily observable as the oral care composition (1) was sufficiently transparent to allow the visibility of the petals of the jasmine. The suspending oral care composition (2) exhibited an even distribution of the petals of the jasmine throughout. Particularly, the inclusion of a thickener, namely xanthum gum, into the oral care composition (2) provided a generally uniform distribution of the petals throughout the suspending oral care composition (2).

The present disclosure has been described with reference to exemplary implementations. Although a limited number of implementations have been shown and described, it will be appreciated by those skilled in the art that changes may be made in these implementations without departing from the principles and spirit of the preceding detailed description. It is intended that the present disclosure be construed as including all such modifications and alterations insofar as they come within the scope of the appended claims or the equivalents thereof.

1. An oral care composition, comprising: an orally acceptable vehicle, and parts of a botanical.

2. The oral care composition of claim 1, wherein a genus of the botanical is selected from Origanum, Thymus, Lavandula, Salvia, Melissa, Cuminum, Pelroselinum, Calendula, Tageles, Boswellia, Sambucus, Copaifera, Curcuma, Allium, Symphylum, Punica, Eulerpe, Sophora, Rheum, Fagopyrum, Camellia, Coplis, Hydraslis, Mahonia, Phellodendron, Berberis, Xanthorhiza, Lonicera, Vaccinium, Cinnamomum, VlZlS, Terminalia, Pinus, Albizia, Melia, Salvadora, Paullinia, Piper, Syzygium, Commiphora, Juglans, Sculellaria, Magnolia, and combinations thereof.

3. The oral care composition of claim 1, wherein the botanical comprises a botanical source selected from roses, gardenias, jasmine, calendula, honeysuckle, cedar wood, aspen bark, willow bark, brahmi, green tea, grapes, pomegranate, echinacea, Centella Asiatica, elderflower, Irish moss, mallow, soap bark, yucca, clary sage, oregano, thyme, and combinations thereof.

4. The oral care composition of claim 3, wherein the botanical source comprises gardenias.

5. The oral care composition of claim 3, wherein the botanical source comprises jasmine.

6. The oral care composition of claim 1, wherein the oral care composition is a mouthwash.

7. The oral care composition of claim 1, wherein the orally acceptable vehicle comprises a thickener configured to at least partially distribute or suspend the parts of the botanical throughout the orally acceptable vehicle.

8. The oral care composition of claim 7, wherein the thickener comprises xanthum gum.

9. The oral care composition of claim 1, wherein the orally acceptable vehicle is free of any thickeners.

10. The oral care composition of claim 1, wherein the parts of the botanical comprise one or more of the following: leaves, fruits, flowers, seeds, petals, or combination thereof.

11. The oral care composition of claim 1, wherein the parts of the botanical comprise petals.

12. A method for preparing the oral care composition of claim 1, the method comprising contacting the parts of the botanical with the orally acceptable vehicle.

13. The method of claim 12, further comprising at least partially distributing the parts of the botanical throughout the orally acceptable vehicle, optionally, wherein at least partially distributing comprises adding a thickener to the orally acceptable vehicle, further optionally, the thickener comprises xanthum gum.

14. A method for treating or reducing one or more oral conditions in a human in need thereof, the method comprising contacting the oral care composition of claim 1 with surfaces of an oral cavity of the human.

15. The method of claim 14, further comprising contacting the oral care composition with the surfaces of the oral cavity at least once per day, optionally, at least twice per day.
